(19) United States
(12) Patent Application Publication
(10) Pub. No.: US 2010/0076319 A1
(43) Pub. Date: Mar. 25, 2010

(54) Pathlength-Corrected Medical Spectroscopy

(75) Inventors: Paul Mannheimer, Danville, CA (US); Martin Debreczeny, Danville, CA (US)

Correspondence Address: NELLCOR PURITAN BENNETT LLC;ATTN: IP LEGAL, 6135 Gunbarrel Avenue, Boulder, CO, 80301, US

(73) Assignee: Nellcor Puritan Bennett LLC, Boulder, CO (US)

(21) Appl. No.: 12/237,539

(22) Filed: Sep. 25, 2008

(51) Int. Cl. A61B 6/00

(52) U.S. Cl. 600/476

(57) ABSTRACT

Systems and methods for reducing scattering effects and correcting for patient to patient anatomical variability are provided. The scattering coefficient of an individual patient's tissue may be corrected for by examining the DC light levels of light passing through the tissue. By comparing the intensity of the light leaving the emitter with the light that reaches the detector to generate a DC component of the signal, which is representative of the anatomical structures of a patient, the AC component of the light may be corrected for the scattering coefficient of the tissue. By correcting the AC signal to account for the scattering coefficient of an individual patient's tissue, a medical sensor may be calibrated in situ for every patient.

### BACKGROUND

The present disclosure relates generally to the field of medical devices and, more particularly, to a system and method generating and processing spectroscopic medical device data.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Spectroscopy may be employed to ascertain the existence and/or concentration of component chemicals in a sample. To perform a spectroscopic analysis on a sample, a source may first send electromagnetic radiation through the sample. The spectrum of electromagnetic radiation that passes through the sample may indicate the absorbance and/or scattering of various constituent components of the sample. Based on the amount and spectrum of the sample absorbance, the presence and/or concentration of distinct chemicals may be detected by employing methods of spectrographic data processing.

Medical spectroscopy employs these techniques to analyze samples from patients for various physiological constituents of interest. For example, pulse oximetry is a technique that may be used to measure various blood flow characteristics, such as the blood-oxygen saturation of hemoglobin in arterial blood, the volume of individual blood pulsations supplying the tissue, and/or the rate of blood pulsations corresponding to each heartbeat of a patient. Pulse oximeters typically utilize a non-invasive sensor that transmits light through a patient's tissue and that photoelectrically detects the absorption and/or scattering of the transmitted light in such tissue. One or more of the above physiological characteristics may then be calculated based upon the amount of light absorbed or scattered. More specifically, the light passed through the tissue is typically selected to be of one or more wavelengths that may be absorbed or scattered by the blood in an amount correlative to the amount of the blood constituent present in the blood. The amount of light absorbed and/or scattered may then be used to estimate the amount of blood constituent in the tissue using various algorithms.

In determining the concentration of the blood constituent, pulse oximetry techniques typically do not compensate for tissue variability between patients. Because the light emitted by a pulse oximetry sensor travels through a heterogeneous sample (i.e., human tissue containing skin, nails, bone, blood, muscle, and nerves), there are many opportunities for the emitted light to be scattered upon contact with the various components found in the tissue sample. The intensity of light transmitted through a patient tissue is a function of the scattering coefficient of both changing and non-changing components. The nonchanging components may be thought of as anatomical structures, such as bone and skin, which do not change significantly over short periods of time. However, the volume and rate of blood flowing in the tissue may change. The transmitted light therefore includes a non-changing DC component that varies slowly with time and represents the effect of the fixed components on the light transmission as well as pulsatile AC component, which varies more rapidly with time and represents the effect that changing tissue blood volume has on the light. Because the attenuation produced by the DC components does not contain information about pulse rate and arterial oxygen saturation, the AC signal is generally used in algorithms to determine the blood oxygen saturation.

### BRIEF DESCRIPTION OF THE DRAWINGS

Advantages of the disclosure may become apparent upon reading the following detailed description and upon reference to the drawings in which:

FIG. 1 is a perspective view of a spectroscopic patient monitor and sensor in accordance with an embodiment;

FIG. 2 is a block diagram of an exemplary patient monitor and sensor in accordance with an embodiment;

FIG. 3 is a flowchart illustrating a method of correcting for tissue scattering effects in a signal generated by a patient sensor in accordance with an embodiment;

FIG. 4 is a block diagram of a method of manufacturing a sensor in accordance with an embodiment

### DETAILED DESCRIPTION

One or more embodiments are described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Provided herein are sensors, systems, and methods for medical spectroscopy that reduce or correct for individual scattering effects of patient tissue. Light that passes through a patient's tissue may be attenuated as it is absorbed and/or scattered by various elements of the tissue. Some of these elements, such as the blood, have a pulsatile nature, while other elements, such as the bone or skin, are generally unchanging over time. Accordingly, part of the light that reaches the detector has a pulsatile component, the AC component, and part of the light that reaches the detector has a generally unchanging component, the DC component. Both components are susceptible to wavelength-dependent scattering and patient-to-patient variations in anatomy.

Although some techniques partially compensate for the scattering effect in the AC and DC components that may vary as a function of an individual patient's anatomy, this is typically accomplished by a constant empirical correction or calibration factor that does not fully correct for scattering. Further, these calibration factors are generally estimates of a larger population and do not account for patient to patient variability.

The light attenuation is related to the scattering coefficient of the patient's tissue, which may vary from patient to patient. The scattering coefficient of an individual patient's tissue may be corrected by examining the DC light levels of light passing through the tissue. Light that leaves a light emitter at a particular wavelength has an intensity that is dependent on the characteristics of the emitter. After passing through the tissue, this light impinges the detector at a reduced intensity. By comparing the intensity of the light leaving the emitter with intensity of the light that reaches the detector to generate a DC component of the signal, which is representative of the anatomical structures of a patient, the AC component of the light may be corrected for the scattering coefficient of the tissue. Because the AC component of the signal provides information about the pulsatile components, this part of the signal may be used to determine physiological characteristics related to pulsatile elements, such as blood constituents. By correcting this AC signal to account for the scattering coefficient of an individual patient's tissue, a medical sensor may be calibrated in situ for every patient.

The present techniques may include a sensor with improved geometry of the light emitting elements and the light detecting components. In an embodiment, sensors are provided in which the light emitting and light detecting components of the sensor are separated from each other to minimize tissue scattering effects that vary from patient to patient. While scattering is wavelength dependent, there are certain emitter/detector separation distances for which changes in the scattering coefficient of a patient's tissue have a reduced effect on the detected intensity. In other words, the sensor geometry may mask larger differences in patient-to-patient tissue variability.

It is envisioned that the disclosed embodiments may be implemented in conjunction with any suitable medical spectroscopic technique. For example, in certain embodiments, the present techniques may be used in conjunction with pulse oximetry, capnography, and/or aquametry (ie., tissue hydration measurements).

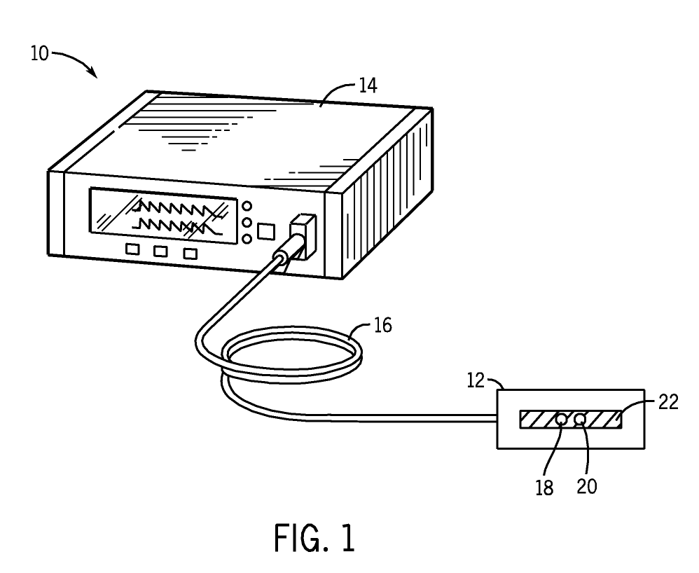

Turning now to FIG. 1, an example of a medical monitoring system that may benefit from the present techniques is depicted. The system 10 of this embodiment includes a physiological sensor 12 that may be attached to a patient. The sensor 12 may generate an output signal based on a monitored physiological characteristic and transmit the output signal to a patient monitor 14. In the depicted embodiment, the sensor 12 is connected to the patient monitor 14 via a cable 16 suitable for transmission of the output signal as well as any other electrical and/or optical signals or impulses communicated between the sensor 12 and monitor 14. In accordance with aspects of the present technique, the sensor 12 and/or the cable 16 may include or incorporate one or more integrated circuit devices or electrical devices, such as a memory, processor chip, or resistor, that may facilitate or enhance communication between the sensor 12 and the patient monitor 14. Likewise the cable 16 may be an adaptor cable, with or without an integrated circuit or electrical device, for facilitating communication between the sensor 12 and various types of monitors, including older or newer versions of the patient monitor 14 or other physiological monitors. In other embodiments, the sensor 12 and the patient monitor 14 may communicate via wireless means, such as using radio, infrared, or optical signals. In such embodiments, a transmission device (not shown) may be connected to the sensor 12 to facilitate wireless transmission between the sensor 12 and the patient monitor 14.

In one embodiment, the patient monitor 14 may be a suitable pulse oximeter, such as those available from Nellcor Puritan Bennett Incorporated. In other embodiments, the patient monitor 14 may be a monitor suitable for measuring other physiological characteristics (such as tissue water fraction, tissue or blood carbon dioxide levels, and so forth) using spectrophotometric or other techniques. Furthermore, the monitor 14 may be a multi-purpose monitor suitable for performing pulse oximetry and/or other physiological and/or biochemical monitoring processes using data acquired via the sensor 12.

As noted above, the data provided to the monitor 14 is generated via the sensor 12. In the example depicted in FIG. 1, the sensor 12 is an exemplary spectrophotometry sensor (such as a pulse oximetry sensor or probe) that includes an emitter 18 and a detector 20 which may be of any suitable type. For example, the emitter 18 may be one or more light emitting diodes adapted to transmit one or more wavelengths of light, such as in the red to infrared range, and the detector 20 may be a photodetector, such as a silicon photodiode package, selected to receive light in the range emitted from the emitter 18. In an embodiment, the emitter 18 and detector 20 may be disposed on a sensor body that may include a surround 22 that is generally dark in color. Such a surround, in an embodiment, may absorb scattered light not first passing through the tissue, which may reduce inaccuracies of measured light at the detector 20. In the depicted embodiment, the sensor 12 is coupled to a cable 16 through which electrical and/or optical signals may be transmitted to and/or from the emitter 18 and detector 20. The sensor 12 may be configured for use with the emitter and detector on the same side of the sensor site (i.e., as a “reflectance type” sensor) or on opposite sides of the sensor site (i.e., as a “transmission type” sensor). During operation, the emitter 18 shines one or more wavelengths of light through the patient's fingertip, or other tissue, and the light received by the detector 20 is processed to determine one or more physiological characteristics of the patient.

In one implementation of the present technique, the emitter 18 and the detector 20 are spaced apart at a distance at which scattering effects are reduced or minimized. In such an implementation, one or more of the sensor 12 and/or cable 16 may be configured to communicate to the monitor 14 that the sensor 12 is a sensor with such geometry. As noted above, in an embodiment, the sensor 12 may have a certain separation between the emitter 18 and the detector 20. In an embodiment, a sensor 12 is provided in which distance between the emitter 18 and detector 20 is greater than 2 mm and less than 5 mm. For example, the distance between the emitter 18 and detector 20 may be about 3 mm. Such a sensor 12 may be configured to operate in a reflectance or transmission configuration. For example, in a reflectance configuration, as depicted in FIG. 1, the emitter 18 and the detector 20 may be spaced side by side with the appropriate separation. In a transmission configuration, the sensor 12 may be configured to be invasive, minimally invasive, such that the emitter 18 and detector 20 capture 2 mm-5 mm of tissue between them. Such a transmission sensor 12 may be a microcaliper or a microneedle configuration in an embodiment. Further, the emitter/detector spacing appropriate for a transmission sensor 12 may be slightly different than for a reflectance sensor. The emitter/detector spacing for a transmission sensor 12 may be determined empirically in one embodiment.

For pulse oximetry applications the oxygen saturation of the patient's arterial blood (SaO₂) may be determined using two or more wavelengths of light emitted by the emitter 18, most commonly red and near infrared wavelengths. After passage through the patient's tissue, a portion of the light emitted at these wavelengths is detected by the detector 20. The detector generates one or more signals, such as electrical or optical signals, in response to the amount of each wavelength that is detected at a given time The generated signals may be digital or, where acquired as analog signals, may be digitized in implementations where digital processing and manipulation of the signals is employed. Such digitalization may be performed at the monitor 14 or prior to reaching the monitor 14. The signals, as noted above, may be transmitted via the cable 16 to the monitor 14, where the oxygen saturation or other physiological characteristic is calculated based on the signals. According to an embodiment, the detector 20 may generate one or more signals that contain AC and DC components of detected light. The DC components may be further processed to calculate a ratio of DC red to DC infrared. The oxygen saturation calculation may be made based at least in part of the ratio.

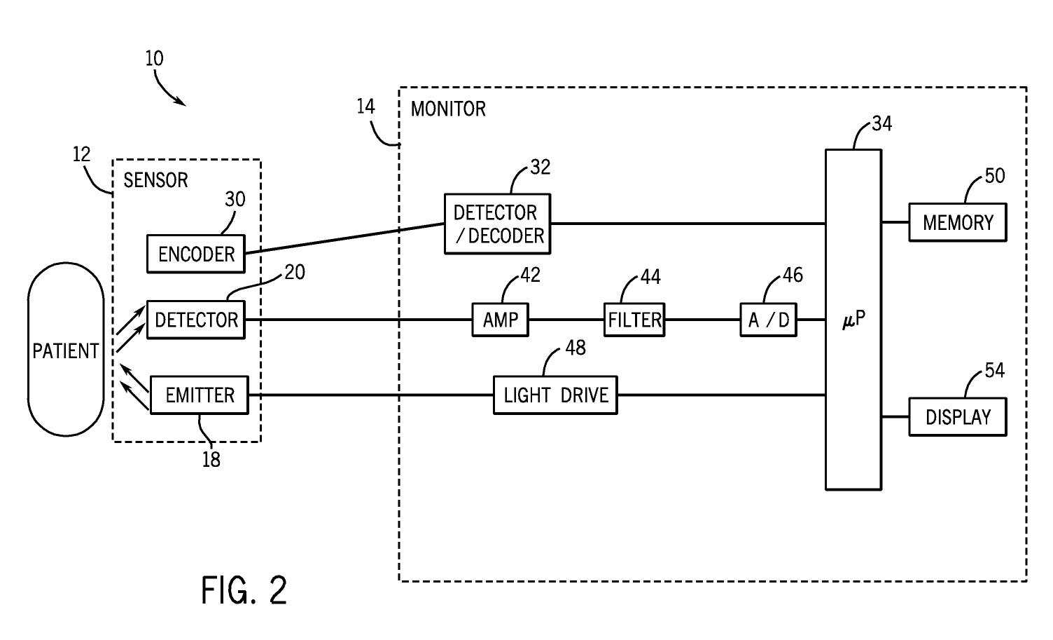

Referring now to FIG. 2, a box-diagram setting forth certain details of the exemplary spectroscopic medical system 10, for example a pulse oximeter, is provided. In particular, a processing component 34 is depicted which is configured to receive a light signal from the sensor 12. The received signal from the detector 20 may be passed through an amplifier 42, a low pass filter 44, and an analog-to-digital converter 46. The processing component 34 may be a general or special purpose processor or circuit suitable for incorporation into the desired structure, such as sensor 12 and/or cable 16 as discussed above with regard to FIG. 1. Likewise, the processing component 34 may be a general or special purpose processor incorporated in the monitor 14.

The processor component 34 may execute code or routines stored in a memory component 50 to accomplish the scattering correction. The memory component 50 may be within the same device or structure as the processing component 34 or may be within a different structure or device in communication with the processing component 34. Such memory components 50 may include solid state or integrated circuit type memory devices or other suitable memory devices, such as magnetic or optical media and/or drives suitable for use in the desired structure or device. The monitor 14 may also include a display 54 on which information about the physiological parameters may be viewed.

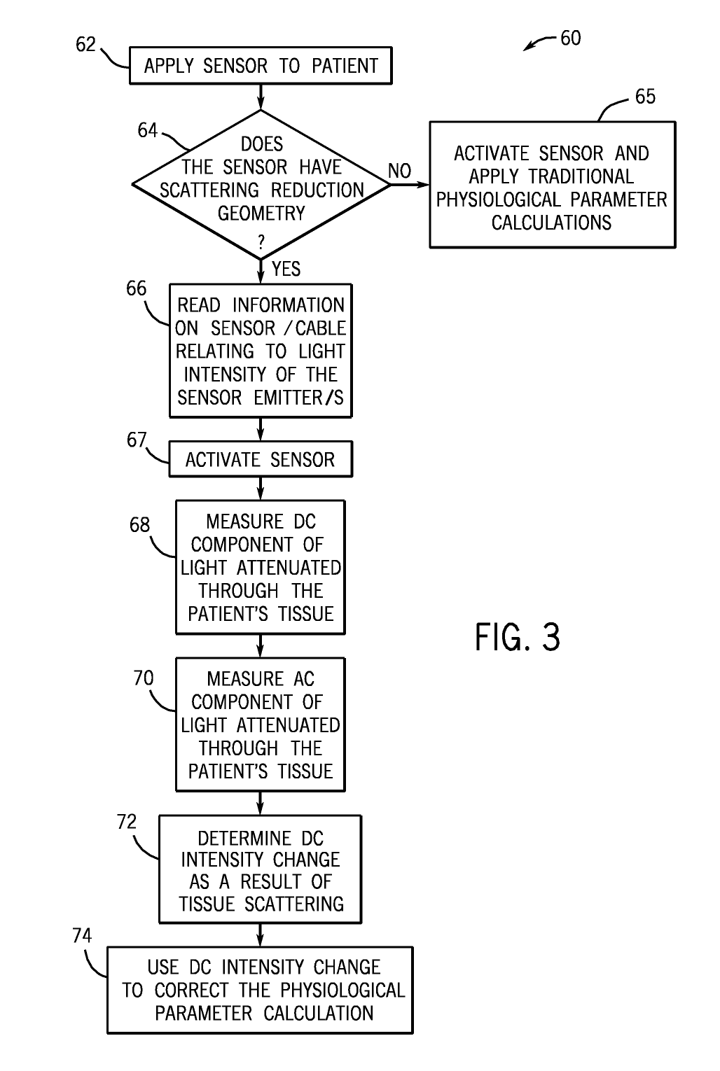

The sensor 12 depicted above in FIG. 1 and FIG. 2 may be used obtain pulse oximetry measurements that may be connected for wavelength and tissue-dependent pathlength variation in the detected light. FIG. 3 is a flowchart depicting a method 60 for using a sensor 12 configured to provide information to allow a compatible monitor 14 to correct for individual patient tissue scattering. In step 62, the sensor 12 is applied to a patient and is driven by the patient monitor 14. In step 64, the processing component 34 determines if the sensor 12 has an emitter/detector spacing associated with reduced scattering effects. For example, the sensor 12 and/or the associated sensor cable 16 may include an encoder 30 and the monitor 14 may include a decoder 32 that reads information encrypted on the encoder 30. From this information, the monitor 14 may determine if the sensor 12 has the appropriate geometry and execute either step 65, which involves applying traditional computations to the waveform signal received form the standard sensor, or steps 66-74, which involve applying certain calculations to the waveform signal generated by a scattering reduction sensor 12.

In step 65, if the encoder 30 indicates that the sensor 12 does not include an emitter/detector spacing associated with reduced scattering effects, the process moves on to activating the sensor 12 and applying certain processing algorithms or calculations. In one embodiment, in step 65 standard pulse oximetry processing algorithms may be employed in which a ratio of light measurements at a red wavelength and at an infrared wavelength may be determined, based in part on which an oxygen saturation and pulse rate may subsequently be determined according to any suitable technique.

However, if the sensor 12 communicates to the monitor 14 that the emitter/detector configuration is associated with reduced scattering effects, the process moves on to execute step 66, which involves reading information from the encoder 30 that provides the intensity of the light emitted at each wavelength. This information may be programmed onto any suitable memory device during the manufacturing process. For example, for emitters that include multiple light emitting elements (each one specific to a certain wavelength), the information encrypted on the encoder may be a separate intensity value for each emitter, or may be a combined number, such as a ratio of the intensities at two different wavelengths. This information may be read at any point while the sensor is connected to the monitor 14.

In addition, at step 67, the sensor 12 is activated by the monitor 14, and measurements are obtained at the monitor via the detector 20. The measurements may include, at step 68, a measurement of the DC component of the light attenuated through the patient's tissue, and at step 70, a measurement of the AC component of the light attenuated through a patient's tissue. Again, in certain medical spectroscopy techniques that use multiple wavelengths of light, the DC light levels obtained in step 68 may be combined into a ratio. For example, in an embodiment, pulse oximetry, a ratio of the DC red intensity and the DC infrared intensity may be obtained.

A change in the DC intensity may be determined at step 72. In an embodiment, for pulse oximetry applications, measuring the DC light levels transmitted through the patient's tissue in the red and near infrared may correct for wavelength-dependent variation in the mean photon pathlength. Photon diffusion theory predicts that variations in the reduced scattering coefficient μ′ₛ and variations in the absorbance coefficient, μₐ, of tissue would affect the mean photon pathlength <l> in different ways, as follows:

$$〈 l 〉 ∝ \sqrt{\frac{{μ}_{s}^{′}}{{μ}_{a}}} . \qquad ( 1 )$$

In contrast, photon diffusion theory predicts that variations in the absorption and reduced scattering coefficients will affect measurements of the DC light intensity, I, transmitted through the tissue, in the same manner:

log(I)∝\sqrt{μₐ·μ′ₛ} (2).

Equations 1 and 2 predict that measurements of the DC light transmitted through the tissue could be used to account for either variations in the absorbance coefficient or the scattering coefficient, but not both simultaneously.

$$\begin{matrix}\frac{log ( {I}^{red} )}{log ( {I}^{IR} )} = \sqrt{\frac{{μ}_{a}^{red} \cdot {μ}_{s}^{′ red}}{{μ}_{a}^{IR} \cdot {μ}_{s}^{′ IR}}} & ( 3 ) \\ 〈 \frac{{l}^{red}}{{l}^{IR}} 〉 = \sqrt{\frac{{μ}_{a}^{IR} \cdot {μ}_{s}^{′ red}}{{μ}_{a}^{red} \cdot {μ}_{s}^{′ IR}}} . & ( 4 )\end{matrix}$$

In an embodiment, in conjunction with a sensor 12 with geometry that minimizes the effect of changes in the scattering coefficient on the transmitted DC light intensity, measurement of changes in the intensity may be used to predict changes in the path length ratio. If the effect of changes in the scattering coefficient on the DC light intensity is small enough to be ignored, the effect of changing the absorption coefficient on the mean photon path length may be directly measured from the DC light intensity. This may be demonstrated by comparing the derivative of the DC light intensity with respect to the absorbance coefficient:

$$Δ log ( I ) = \frac{\partial log ( I )}{\partial {μ}_{a}} = \frac{1}{2} \sqrt{\frac{{μ}_{s}^{′}}{{μ}_{s}}} Δ {μ}_{a} . \qquad ( 5 )$$

The derivation of the mean photon path length with respect to the absorbance coefficient is presented in Equation (6), below:

$$Δ 〈 l 〉 = \frac{\partial 〈 l 〉}{\partial {μ}_{a}} = - \frac{1}{2 {μ}_{a}} \sqrt{\frac{{μ}_{s}^{′}}{{μ}_{a}}} {Δμ}_{a} . \qquad ( 6 )$$

Combining equations 5 and 6, for red and IR wavelengths results in:

$$\frac{Δ 〈 {l}^{red} 〉}{Δ 〈 {l}^{IR} 〉} = \frac{Δ log ( {I}^{red} )}{Δ log ( {I}^{IR} )} \cdot C . \qquad ( 7 )$$

Equation 7, which uses the change in the DC intensity of step 72, demonstrates that a change in the average relative path length traveled by photons at two different wavelengths can be compensated by measuring the relative change in the absorption of light by the tissue at the two wavelengths. The correction term C in Equation 7 may be determined either theoretically or by empirical calibration. For example, the theoretical value of C may be determined from

$$\frac{{μ}_{a}^{IR}}{{μ}_{a}^{red}} ,$$

using estimated values that are typical for the tissue being optically interrogated. Alternatively, the value of C may be determined empirically by comparing non-invasive optical measurements with invasive arterial blood oxygen measurements from human subjects. By this method, the value of C is set so that error is minimized between the oxygenation computed optically and the oxygenation measured invasively. Whether estimated by theoretical or empirical means, deviations in the relative tissue absorption coefficients from the average case may be compensated by measuring the relative DC absorption according to Equation 7.

From the change in DC intensity determined in step 72 and the AC component of the signal measured in step 70, a physiological parameter may be determined in step 74. The change in the DC intensity may be used as a correction factor to account for the tissue scattering in the AC component of the signal by using the corrected mean photon pathlength ratio, which is provided in Equation 7 in the saturation calculation. In one embodiment, the change in DC intensity may be related to a series of calibration curves. For a particular change in DC intensity, a calibration curve may be selected and the AC component of the signal may fitted to the curve.

In another embodiment the AC components may be corrected by using the change in mean photon pathlength ratio calculated from the change in the DC intensity ratio. In one embodiment, the change in mean photon pathlength ratio can be applied to the calculation of R, which is equal to a ratio of the pulsatile red component divided by the steady-state red component, divided by the same ratio of the pulsatile and the steady state JR components, and which may be used to determine a patient's oxygen saturation. For example, below equation 8 represents the typical saturation calculation for the pulsatile factor R using a mean photon pathlength ratio that is estimated from empirical studies using a healthy pool of volunteers. The mean photon pathlength ratio is used as a correction factor for every patient, regardless of individual patient variability.

$$R = \frac{{μ}_{a}^{red}}{{μ}_{a}^{IR}} \cdot \frac{〈 {l}^{red} 〉}{〈 {l}^{IR} 〉} \qquad ( 8 )$$

In contrast, the present disclosure provides for a corrected mean photon pathlength ratio that may be determined for every individual patient. After determining the change in DC intensity in step 72, the intensity change of DC component is used to determine the change in mean photon pathlength in Equation 7. The change in mean photon pathlength ratio may be used to perform a corrected calculation of a pulsatile factor R′ in Equation 9. The corrected version of this equation involves using a corrected mean photon pathlength ratio as a calibration factor for the ratio of ratios.

$${R}^{′} = \frac{{μ}_{a}^{red}}{{μ}_{a}^{IR}} \cdot \frac{〈 {l}^{′ red} 〉}{〈 {{l}^{′ }}^{IR} 〉} \qquad ( 9 )$$

The corrected mean photon pathlength ratio may be directly determined from the change in the mean photon pathlength ratio of Equation 7 by a simple calculation (e.g., by using a multiplier) or by correlating the change in mean photon pathlength to a previously determined value via curve fitting or a look-up table. Upon determining the corrected R′ value based on the corrected mean photon pathlength, the corrected R′ value may be used to determine an oxygen saturation value.

In a pulse oximetry sensor 12 in which an emitter 18 may include two light emitting elements, the light emitting elements may have a characteristic emitted light intensity. Generally, two light emitting elements, one red and one infrared, are paired to form an emitter 18. Their characteristic intensities may be thought of as a ratio. For example, where the red light emitting element is twice as bright as the infrared light emitting element, the ratio of I^{RED}/I^{IR} would be 2. The intensity ratio of the emitter pair is used as a starting point for determining the change in intensity of the DC component after the light has passed through the tissue. For example, the light hitting the detector 20 may be normalized for the brightness difference between the light emitters.

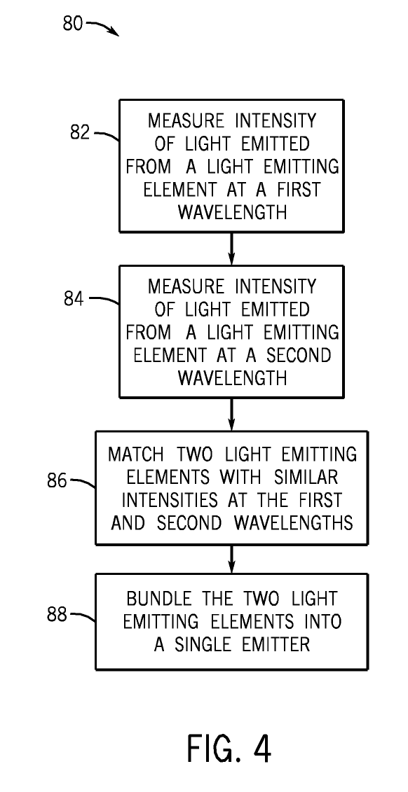

In certain embodiments, it may be advantageous to provide sensors 12 that are designed with light emitters 18 that have a certain intensity or relative intensity. In such an embodiment, where the ratio of I^{RED}/I^{IR} is 1, the normalization step to account for the difference in brightness between red and IR light emitting elements may be omitted. In addition, it may be advantageous to know the starting brightness of the light emitting elements in order to calibrate a sensor 12 against a healthy population. If the sensor 12 with matched light emitting elements is used to calibrate a healthy population, the change in DC intensity from a similarly matched sensor 12 may be directly compared to a table or graph of results from the healthy population without first normalizing the brightness levels to the brightness levels of the sensors used to calibrate the population. In such an embodiment, the pathlength correction may be a simple multiplier to the mean pathlength calibration. FIG. 4 is a block diagram of a method 80 of manufacturing a sensor 12 as provided herein. In such an embodiment, the correction for the change in DC intensity may be less complex if the relative intensities of the light emitting elements of a sensor 12 are close to equal. An emitter 18 may have one or more light emitting elements, each specific for a particular wavelength. In an embodiment, the light emitting elements of an emitter 18 may have light intensities that are relatively close to one another. This may be accomplished during the sensor manufacturing process by measuring the emitted light intensity of each light emitting element at each wavelength. For example, in an embodiment for manufacturing a sensor with an emitter that includes two light emitting elements, the intensity of light emitted by individual light emitters at a first wavelength may be measured (block 82) along with the intensity of light emitted by individual light emitters at a second wavelength (block 84). The light emitters of the first wavelength may be graded, ranked, marked, or separated according to their various intensities. The light emitters of the second wavelength may be similarly separated so that a matching process (block 86) may occur in which a light emitting element of the first wavelength is matched with a light emitting element of the second wavelength according to their respective intensities. For example, the matching may be accomplished according to a desired ratio that may be, in an embodiment, close to 1. In an embodiment, the ratio of the intensity of the light emitting components may be in a range of 0.5 to 1.5 such as 0.9 to 1.1.

When two light emitting elements, one of each of the two wavelengths, are matched, based on their respective intensities, they may be placed in any suitable emitter housing to form an emitter 18. The emitter 18 in turn may be disposed on a sensor body along with a compatible detector to form a sensor 12.

In an embodiment, the method 80 may be implemented with emitters 18 that include any number of light emitting elements. For example, in an embodiment, in a sensor 12 that includes an emitter 18 that emits three wavelengths of light, the relative intensities of each of the three light emitting elements may be matched. Further, the emitter 16 may be one or more light emitting diodes adapted to transmit one or more wavelengths of light in the red to infrared range, and the detector 18 may one or more photodetectors selected to receive light in the range or ranges emitted from the emitter 16. Alternatively, an emitter 16 may also be a laser diode, tunable laser, or a vertical cavity surface emitting laser (VCSEL), or other light source. The emitter 16 and detector 18 may also include optical fiber sensing elements.

In an embodiment, an emitter 16 may include a broadband or “white light” source, and the detector could include any of a variety of elements for selecting specific wavelengths, such as reflective or refractive elements or interferometers. These types of emitters and/or detectors may be coupled to the rigid or rigidified sensor via fiber optics.

In an embodiment, a sensor 12 may sense light detected from the tissue at a different wavelength from the light emitted into the tissue. Such sensors may be adapted to sense fluorescence, phosphorescence, Raman scattering, Rayleigh scattering, and/or multi-photon events or photoacoustic effects. For pulse oximetry applications using either transmission or reflectance type sensors the oxygen saturation of the patient's arterial blood may be determined using two or more wavelengths of light, most commonly red and near infrared wavelengths. Similarly, in other applications, a tissue water fraction (or other tissue constituent related metric) or a concentration of one or more biochemical components in an aqueous environment may be measured using two or more wavelengths of light. In various embodiments, these wavelengths may be infrared wavelengths between about 1,000 nm to about 2,500 nm.

It should be understood that, as used herein, the term “light” may refer to one or more of ultrasound, radio, microwave, millimeter wave, infrared, visible, ultraviolet, gamma ray or X-ray electromagnetic radiation, and may also include any wavelength within the ultrasound, radio, microwave, millimeter wave, infrared, visible, ultraviolet, or X-ray spectra, and that any suitable wavelength of light may be appropriate for use with the present techniques.

In one embodiment, the sensor 12 may be initially applied to a reflective substrate or reflective material in order to determine the relative intensities of the light emitted at two (or more) wavelengths. Such an embodiment may represent a calibration or initialization step for the sensor 12. As such calibration may be independent of patient application, this step may be done by the manufacturer or by a healthcare provider, for example through prompting by a monitor when the sensor 12 is applied to the monitor. After calibration, the information relating to the intensity or relative intensity may be stored on a sensor memory or by the monitor for further processing.

While the above disclosure may be susceptible to various modifications and alternative forms, various embodiments have been shown by way of example in the drawings and have been described in detail herein. However, it should be understood that the claims are not intended to be limited to the particular forms disclosed. Rather, the claims are to cover all modifications, equivalents, and alternatives falling within the spirit and scope of the disclosure. Particularly, it should be noted that the techniques described may be utilized individually or in any combination. Moreover, the steps of the techniques described may be performed in various orders other than the order recited with reference to each figure, as will be appreciated by those of ordinary skill in the art.

What is claimed is:

1. A sensor comprising:
an emitter capable of transmitting one or more wavelengths of light of one or more intensities;
a detector capable detecting the one or more wavelengths of light,
wherein the emitter and the detector being positioned a distance of 2 mm-5 mm apart; and
a memory associated with the sensor, wherein the memory comprises data relating to the one or more intensities of the one or more wavelengths of light.

2. The sensor of claim 1, wherein the sensor comprises a pulse oximetry sensor or an aquametry sensor.

3. The sensor of claim 1, wherein the substrate comprises a substantially dark area substantially surrounding the light emitting element and the detector.

4. The sensor of claim 1, wherein the memory is associated with a cable sensor operatively connected to the sensor.

5. The sensor of claim 1, wherein the emitter is capable of emitting light of a first wavelength at a first intensity and light of a second wavelength at a second intensity.

6. The sensor of claim 5, wherein the first intensity and the second intensity are substantially the same.

7. The sensor of claim 1, wherein the memory comprises identification data relating to the distance between the emitter and the detector.

8. A physiological monitor comprising:
a processor programmed to:
read information on a memory associated with a sensor about one or more intensities of one or more wavelengths of light emitted by an emitter associated with the sensor;
receive a signal from the sensor, wherein the signal comprises a measured AC component and a measured DC component of the one or more wavelengths of light attenuated through a patient's tissue;
determine a change in intensity of the DC component based on the intensity of the one or more wavelengths of light emitted and the measured DC component of the one or more wavelengths of light; and
determine a physiological parameter based at least in part on the AC component and the change in intensity of the DC component.

9. The monitor of claim 8, comprising the sensor, wherein the sensor comprises a detector spaced about 2-5 mm away from the emitter.

10. The monitor of claim 8, wherein the information on the memory comprises information about a first intensity of light emitted at a first wavelength and a second intensity of light of emitted at a second wavelength.

11. The monitor of claim 10, wherein the change in intensity of the DC component is determined based in part on a ratio of the measured DC component at the first wavelength and the second wavelength.

12. The monitor of claim 8, wherein the monitor comprises a pulse oximetry monitor.

13. A method comprising:
reading information on a memory associated with a sensor about one or more intensities of one or more wavelengths of light emitted by an emitter associated with the sensor;
receiving a signal from the sensor, wherein the signal comprises a measured AC component and a measured DC component of the one or more wavelengths of light attenuated through a patient's tissue;
determining a change in intensity of the DC component based on the one or more intensities of the one or more wavelengths of light emitted and the measured DC component of the one or more wavelengths of light; and
determining a physiological parameter based at least in part on the AC component and the change in intensity of the DC component.

14. The method of claim 13, reading information on the memory about a relative spacing of the emitter and a detector associated with the sensor.

15. The method of claim 13, wherein reading the information on the memory comprises reading information about a first intensity of light emitted at a first wavelength and a second intensity of light of emitted at a second wavelength.

16. The method of claim 13, wherein determining the change in intensity of the DC component comprises determining a ratio of the measured DC component at the first wavelength and the second wavelength.

17. A method of manufacturing a sensor comprising:
determining a first intensity of light of a first wavelength emitted by a first light emitting element;
determining a second intensity of light of a second wavelength emitted by a second light emitting element, wherein when the ratio of the first intensity and the second intensity is within a certain range, the first light emitting element and the second light emitting element are placed together to form an emitter; and
disposing the emitter and a detector capable detecting the first wavelength of light and the second wavelength of light a distance of 2 mm-5 mm apart on a substrate.

18. The method of claim 18, comprising providing a dark area on the substrate substantially surrounding the emitter and the detector.

19. The method of claim 18, comprising associating a memory with the sensor, wherein the memory comprises data relating to the first intensity and the second intensity.

20. The method of claim 19, wherein the data relating to the first intensity and the second intensity comprises the ratio of the first intensity to the second intensity.
